(19) United States
(12) Patent Application Publication
(10) Pub. No.: US 2019/0183773 A1
(43) Pub. Date: Jun. 20, 2019

(54) COSMETIC AGENT FOR THE TEMPORARY SHAPING OF KERATIN-CONTAINING FIBERS WITH STARCH

(71) Applicant: Henkel AG & Co. KGaA, Duesseldorf (DE)

(72) Inventors: Thorsten Knappe, Schenefeld (DE); Mathias Schriefers, Moenchengladbach (DE)

(73) Assignee: Henkel AG & Co. KGaA, Duesseldorf (DE)

(21) Appl. No.: 16/220,917

(22) Filed: Dec. 14, 2018

(30) Foreign Application Priority Data
Dec. 15, 2017 (DE) 10 2017 222 854.7

(51) Int. Cl. A61K 8/73; A61K 8/81; A61K 8/31; A61Q 5/06; B05B 9/04

(52) U.S. Cl. CPC A61K 8/732; A61K 8/8158; A61K 2800/594; A61Q 5/06; B05B 9/04; A61K 8/31

(57) ABSTRACT

The present disclosure relates to a cosmetic agent containing potato starch for the temporary shaping of keratinic fibers, in particular human hair. In an alternate embodiment, a method of temporarily shaping keratinic fibers is provided. The method includes applying a cosmetic agent to the keratinic fibers, wherein the cosmetic agent comprises potato starch.

### CROSS-REFERENCE TO RELATED APPLICATION

This application claims priority to German Patent Application No. 10 2017 222 854.7, filed Dec. 15, 2017, which is incorporated herein by reference in its entirety.

### TECHNICAL FIELD

The present disclosure relates to a cosmetic agent containing starch for the temporary shaping of keratinic fibers, in particular human hair.

### BACKGROUND

The temporary design of hairstyles for a longer period of up to several days usually requires the application of firming active ingredients. Hair treatment agents which are used for a temporary shaping of the hair therefore play an important role. Corresponding agents for temporary deformation usually contain synthetic polymers and/or waxes as a firming active ingredient. Agents for supporting the temporary shaping of hair can be formulated, for example, as a hair spray, hair wax, hair gel, or hair foam.

The most important property of an agent for the temporary deformation of hair, hereinafter also referred to as a styling agent, is to give the treated fibers in the newly modeled shape, that is, a shape impressed on the hair, as strong and lasting hold as possible. One also speaks of strong hairstyle or the high degree of hold of the styling agent. The hairstyle hold is essentially determined by the type and amount of firming active ingredients used, but an influence of the further components of the styling agent may also be provided.

In addition to a high degree of hold, styling agents must meet a whole range of other requirements. These may be broadly subdivided into properties on the hair, properties of the particular formulation, for example properties of the foam, of the gel or of the sprayed aerosol, and of properties that relate to the handling of the styling agent, wherein the properties on the hair are of particular importance. Particularly noteworthy are moisture resistance, low tack and a balanced conditioning effect. Furthermore, a styling agent should be universally usable for all hair types and mild to hair and skin.

In order to meet the different requirements, a variety of synthetic polymers, which are used in styling agents, have already been developed as firming active ingredients. The polymers can be subdivided into cationic, anionic, nonionic and amphoteric firming polymers. Alternatively or additionally, waxes are used as firming active ingredients. Ideally, the polymers and/or waxes, when applied to hair, provide a polymer film or film which, on the one hand, gives the hairstyle a strong hold but, on the other hand, is sufficiently flexible not to break under stress.

In the context of this application, the terms film-forming polymer and firming polymer are used synonymously.

The synthetic polymers commonly used in agents for temporary shaping are prepared from corresponding synthetically available monomers. Said monomers are obtained from fossil materials such as petroleum by conversion to the corresponding polymer building blocks, among other things, with the expenditure of energy. Since the monomers are already prepared synthetically, the polymers commonly used are fully synthetic polymers. In the context of a more sustainable use of raw materials and energy, it remains desirable to use for cosmetic products only such cosmetic raw materials that are available from renewable raw materials with the least possible use of energy. However, a reduction in quantity or even an exchange of said fully synthetic polymers can only be carried out when the replacement polymers produce the properties desired for the intended use and give the keratin-containing fibers a sufficient, stable hold on the impressed shape.

### BRIEF SUMMARY

Cosmetic agents and methods for temporarily shaping keratinic fibers are provided. In an exemplary embodiment, a cosmetic agent comprises from about 0.1 to about 50% by weight of potato starch, based on a total weight of the cosmetic agent.

A method of temporarily shaping keratinic fibers is provided in another embodiment. The method includes applying a cosmetic agent to the keratinic fibers. The cosmetic agent comprises potato starch at from about 0.1 to about 50 weight percent, based on a total weight of the cosmetic agent.

### DETAILED DESCRIPTION

The following detailed description is merely exemplary in nature and is not intended to limit the disclosure or the application and uses of the subject matter as described herein. Furthermore, there is no intention to be bound by any theory presented in the preceding background or the following detailed description.

The object underlying the present disclosure is to provide a cosmetic agent which is suitable for the temporary shaping of keratin-containing fibers, in which the use of fully synthetic polymers is reduced or in which the use of fully synthetic polymers is dispensed with, wherein the styling properties are to be maintained at a high level.

This object is achieved by a cosmetic agent for the temporary shaping of keratinic fibers, which agent contains from about 0.1 to about 50% by weight, in particular from about 1 to about 20% by weight of potato starch, based on the total weight of the agent.

Cosmetic agents for the temporary shaping of human hair are also referred to as styling agents. In particular, the present disclosure relates to styling agents such as foams, mousses, gels, waxes, pastes, lotions, emulsions or clays. The product form “clay” refers to highly viscous, waxy cosmetic agents which contain, among other things, clay compounds (for example kaolin).

It has surprisingly been found in the context of the present disclosure that by adding potato starch to a cosmetic agent for the temporary deformation of keratinic fibers, in particular of human hair, the amount of fully synthetic polymers as a film former can be reduced, without necessarily sacrificing the styling properties.

It has also been found that, in particular, the stiffness of the polymer film and thereby the hairstyle hold can be greatly increased by adding potato starch to a cosmetic agent containing fully synthetic firming polymers.

Surprisingly, it was found that, contrary to expectations, no negative aspects such as plaque formation were found. If the polymer film is too brittle, it results in the formation of so-called film plaques, that is, residues that detach during the movement of the hair and give the impression that the user of the corresponding styling agent might have dandruff.

Other commonly required properties of cosmetic agents for the temporary shaping of keratinic fibers, such as long-term hold and low tack, still remain in this case.

The term keratinic fibers as contemplated herein comprises furs, wool and feathers, but in particular human hair.

In a further embodiment of the present disclosure, there is a potato starch that is partially or fully modified. The modified potato starch is selected from the group of gelatinized potato starch, alkaline modified potato starch and mixtures thereof.

Potato starch (Solanum tuberosum starch) can physically bind, swell and gelatinize many times its own weight in water under the influence of heat. When heated with water, the starch swells, the layers burst, and at about 62.5° C. produces starch paste.

Particularly preferred according to the present disclosure is when the potato starch is either cooked before use in the agent (gelatinization) or an aqueous dispersion is adjusted strongly alkaline with, for example, NaOH, and thus initiating the swelling. Following this, the alkaline solution of an organic acid, for example lactic acid, can be readjusted to any pH value without preventing further processing.

In principle, the cosmetic agents may further contain natural starch of other species, in particular wheat starch, tapioca starch, rice starch, corn starch and/or horse chestnut starch. When these starches of other species are present, it is particularly preferred to use them in amounts of from about 10% to about 200% by weight, based on potato starch.

The agent preferably contains at least one emulsifier as a further essential component. Suitable emulsifiers are, in principle, anionic, cationic, nonionic and ampholytic surface-active compounds which are suitable for use on the human body. The ampholytic surface-active compounds include zwitterionic surface-active compounds and ampholytes. Preference is given to nonionic emulsifiers and/or ampholytic emulsifiers.

Emulsifiers cause the formation of water- or oil-stable adsorption layers at the phase interface, which layers protect the dispersed droplets against coalescence and thus stabilize the emulsion. Emulsifiers are therefore constructed like surfactants from a hydrophobic molecule part and a hydrophilic molecule part. Hydrophilic emulsifiers preferably form O/W emulsions (oil-in-water—emulsions) and hydrophobic emulsifiers preferably form W/O emulsions (water-in-oil—emulsions). The selection of these emulsifying surfactants or emulsifiers depends on the substances to be dispersed and the particular outer phase and the fineness of the emulsion. Preference is given to O/W emulsifiers, particularly preferred emulsifiers are addition products of from about 15 to about 60 moles of ethylene oxide with castor oil and hydrogenated castor oil, in particular PEG-40 Castor Oil (INCI). The particularly preferred emulsifiers offer the advantage of being able to be obtained from natural, non-fully synthetic raw material sources.

A preferred ampholytic emulsifier is, for example, cocoamidopropylbetaine.

The cosmetic agent may contain the emulsifier in a total amount of from about 0.1 to about 2% by weight, preferably from about 0.25 to about 1.5% by weight, more preferably from about 0.5 to about 1% by weight, based on the total weight of cosmetic agent.

The agents contain the ingredients or active ingredients in a cosmetically acceptable carrier.

Preferred cosmetically acceptable carriers are aqueous, alcoholic or aqueous-alcoholic media having preferably at least about 10% by weight water, based on the total agent. In particular, the lower alcohols having from 1 to 4 carbon atoms usually used for cosmetic purposes, such as, for example, ethanol and isopropanol, can be present as alcohols.

It may be preferred to use at least one (C₁ to C₄) monoalkyl alcohol in the agents, in particular in an amount of from about 1 to about 50% by weight, in particular from about 5 to about 30% by weight. This is, in turn, preferred in particular for packaging as a pump foam or aerosol foam.

Further preferred cosmetic agents contain water as a cosmetic carrier. In these embodiments, the cosmetic agent contains water as the main component. The water content of the cosmetic agents is from about 5 to about 90% by weight, preferably from about 15 to about 80% by weight and more preferably from about 40 to about 75% by weight, based on the total weight of cosmetic agent.

The cosmetic agent may further contain at least one firming polymer other than the potato starch mentioned above. Examples are cationic, anionic, nonionic or amphoteric polymers.

Examples of firming polymers are Acrylamide/Ammonium Acrylate Copolymer, Acrylamides/DMAPA Acrylates/Methoxy PEG Methacrylate Copolymer, Acrylamidopropyltrimonium Chloride/Acrylamide Copolymer, Acrylamidopropyltrimonium Chloride/Acrylates Copolymer, Acrylates/Acetoacetoxyethyl Methacrylate Copolymer, Acrylates/Acrylamide Copolymer, Acrylates/Ammonium Methacrylate Copolymer, Acrylates/t-Butylacrylamide Copolymer, Acrylates Copolymer, Acrylates/C1-2 Succinates/Hydroxyacrylates Copolymer, Acrylates/Lauryl Acrylate/Stearyl Acrylate/Ethylamine Oxide Methacrylate Copolymer, Acrylates/Octylacrylamide Copolymer, Acrylates/Octylacrylamide/Diphenyl Amodimethicone Copolymer, Acrylates/Stearyl Acrylate/Ethylamine Oxide Methacrylate Copolymer, Acrylates/VA Copolymer, Acrylates/VP Copolymer, Adipic Acid/Diethylenetriamine Copolymer, Adipic Acid/Dimethylaminohydroxypropyl Diethylenetriamine Copolymer, Adipic Acid/Epoxypropyl Diethylenetriamine Copolymer, Adipic Acid/Isophthalic Acid/Neopentyl Glycol/Trimethylolpropane Copolymer, Allyl Stearate/VA Copolymer, Aminoethylacrylate Phosphate/Acrylates Copolymer, Aminoethylpropanediol-Acrylates/Acrylamide Copolymer, Aminoethylpropanediol-AMPD-Acrylates/Diacetoneacrylamide Copolymer, Ammonium VA/Acrylates Copolymer, AMPD-Acrylates/Diacetoneacrylamide Copolymer, AMP-Acrylates/Allyl Methacrylate Copolymer, AMP-Acrylates/C1-18 Alkyl Acrylates/C1-8 Alkyl Acrylamide Copolymer, AMP-Acrylates/Diacetoneacrylamide Copolymer, AMP-Acrylates/Dimethylaminoethylmethacrylate Copolymer, Bacillus/Rice Bran Extract/Soybean Extract Ferment Filtrate, Bis-Butyloxyamodimethicone/PEG-60 Copolymer, Butyl Acrylate/Ethylhexyl Methacrylate Copolymer, Butyl Acrylate/Hydroxypropyl Dimethicone Acrylate Copolymer, Butylated PVP, Butyl Ester of Ethylene/MA Copolymer, Butyl Ester of PVM/MA Copolymer, Calcium/Sodium PVM/MA Copolymer, Corn Starch/Acrylamide/Sodium Acrylate Copolymer, Diethylene Glycolamine/Epichlorohydrin/Piperazine Copolymer, Dimethicone Crosspolymer, Diphenyl Amodimethicone, Ethyl Ester of PVM/MA Copolymer, Hydrolyzed Wheat Protein/PVP Crosspolymer, Isobutylene/Ethylmaleimide/Hydroxyethylmaleimide Copolymer, Isobutylene/MA Copolymer, Isobutylmethacrylate/Bis-Hydroxypropyl Dimethicone Acrylate Copolymer, Isopropyl Ester of PVM/MA Copolymer, Lauryl Acrylate Crosspolymer, Lauryl Methacrylate/Glycol Dimethacrylate Crosspolymer, MEA-Sulfite, Methacrylic Acid/Sodium Acrylamidomethyl Propane Sulfonate Copolymer, Methacryloyl Ethyl Betaine/Acrylates Copolymer, Octylacrylamide/Acrylates/Butylaminoethyl Methacrylate Copolymer, PEG/PPG-25/25 Dimethicone/Acrylates Copolymer, PEG-8/SMDI Copolymer, Polyacrylamide, Polyacrylate-6, Polybeta-Alanine/Glutaric Acid Crosspolymer, Polybutylene Terephthalate, Polyester-1, Polyethylacrylate, Polyethylene Terephthalate, Polymethacryloyl Ethyl Betaine, Polypentaerythrityl Terephthalate, Polyperfluoroperhydrophenanthrene, Polyquaternium-1, Polyquaternium-2, Polyquaternium-4, Polyquaternium-5, Polyquaternium-6, Polyquaternium-7, Polyquaternium-8, Polyquaternium-9, Polyquaternium-10, Polyquaternium-11, Polyquaternium-12, Polyquaternium-13, Polyquaternium-14, Polyquaternium-15, Polyquaternium-16, Polyquaternium-17, Polyquaternium-18, Polyquaternium-19, Polyquaternium-20, Polyquaternium-22, Polyquaternium-24, Polyquaternium-27, Polyquaternium-28, Polyquaternium-29, Polyquaternium-30, Polyquaternium-31, Polyquaternium-32, Polyquaternium-33, Polyquaternium-34, Polyquaternium-35, Polyquaternium-36, Polyquaternium-37, Polyquaternium-39, Polyquaternium-45, Polyquaternium-46, Polyquaternium-47, Polyquaternium-48, Polyquaternium-49, Polyquaternium-50, Polyquaternium-55, Polyquaternium-56, Polysilicone-9, Polyurethane-1, Polyurethane-6, Polyurethane-10, Polyvinyl Acetate, Polyvinyl Butyral, Polyvinylcaprolactam, Polyvinylformamide, Polyvinyl Imidazolinium Acetate, Polyvinyl Methyl Ether, Potassium Butyl Ester of PVM/MA Copolymer, Potassium Ethyl Ester of PVM/MA Copolymer, PPG-70 Polyglyceryl-10 Ether, PPG-12/SMDI Copolymer, PPG-51/SMDI Copolymer, PPG-10 Sorbitol, PVM/MA Copolymer, PVP, PVPNA/Itaconic Acid Copolymer, PVP/VA/Vinyl Propionate Copolymer, Rhizobian Gum, Rosin Acrylate, Shellac, Sodium Butyl Ester of PVM/MA Copolymer, Sodium Ethyl Ester of PVM/MA Copolymer, Sodium Polyacrylate, Sterculia Urens Gum, Terephthalic Acid/Isophthalic Acid/Sodium Isophthalic Acid Sulfonate/Glycol Copolymer, Trimethylolpropane Triacrylate, Trimethylsiloxysilylcarbamoyl Pullulan, VA/Crotonates Copolymer, VA/Crotonates/Methacryloxybenzophenone-1 Copolymer, VA/Crotonates/Vinyl Neodecanoate Copolymer, VA/Crotonates/Vinyl Propionate Copolymer, VA/DBM Copolymer, VA/Vinyl Butyl Benzoate/Crotonates Copolymer, Vinylamine/Vinyl Alcohol Copolymer, Vinyl Caprolactam/VP/Dimethylaminoethyl Methacrylate Copolymer, VP/Acrylates/Lauryl Methacrylate Copolymer, VP/Dimethylaminoethylmethacrylate Copolymer, VP/DMAPA Acrylates Copolymer, VP/Hexadecene Copolymer, VP/VA Copolymer, VP/Vinyl Caprolactam/DMAPA Acrylates Copolymer, Yeast Palmitate or Styrene/VP Copolymer.

Siloxanes are also suitable as preferred firming polymers. Homopolyacrylic acid (INCI: Carbomer), which is commercially available under the name Carbopol® in various implementations, is also suitable as a firming polymer.

The firming polymer is preferably selected from vinylpyrrolidone-containing polymers. The firming polymer is particularly preferably selected from the group of polyvinylpyrrolidone, vinylpyrrolidone/vinyl acetate copolymer, vinylcaprolactam/VP/dimethylaminoethyl methacrylate copolymer (INCI), VP/DMAPA Acrylate Copolymer (INCI) and mixtures thereof.

A likewise preferred firming polymer is octylacrylamide/acrylates/butylaminoethyl methacrylate copolymer (INCI), which is sold under the name “Amphomer®” by AkzoNobel.

The cosmetic agents can particularly preferably contain a polymer selected from chitosan and its derivatives as a further firming polymer. Chitosans are biopolymers and are counted among the group of hydrocolloids. Chemically, these are partially deacetylated chitins of different molecular weight.

The production of chitosans is based on chitin, preferably the shell remains of crustaceans, which are available as cheap raw materials in large quantities. The chitin is usually first deproteinized by the addition of bases, demineralized by the addition of mineral acids and finally deacetylated by addition of strong bases, wherein the molecular weights can be distributed over a broad spectrum. Preferably used are those types having an average molecular weight of from about 800,000 to about 1,200,000 daltons, a Brookfield viscosity (about 1% by weight in glycolic acid) below about 5000 mPas, a degree of deacetylation in the range of from about 80 to about 88%, and have an ash content of less than about 0.3% by weight.

In addition to the chitosans, it is also possible to use cationically derivatized chitosan (such as, for example, quaternization products) or alkoxylated chitosan.

Most preferably, the cosmetic agents may contain neutralization products of chitosan with at least one acid as chitosan derivative(s). The acid used for the neutralization is preferably selected from organic acids, in particular from formic acid, acetic acid, citric acid, lactic acid, pyrrolidonecarboxylic acid, nicotinic acid, hydroxyisobutyric acid, hydroxyisovaleric acid or mixtures of these acids. It is very particularly preferred to select the acid(s) used for the neutralization from lactic acid, pyrrolidonecarboxylic acid, nicotinic acid, hydroxyisobutyric acid, hydroxyisovaleric acid or mixtures of these acids.

Suitable chitosan (derivatives) are, for example, freely available commercially under the trade names Hydagen® CMF (about 1% by weight active substance in aqueous solution with about 0.4% by weight glycolic acid, molecular weight from about 500,000 to about 5,000,000 g/mole Cognis), Hydagen® HCMF (chitosan (about 80%) deacetylated), molecular weight from about 50,000 to about 1,000,000 g/mole, Cognis), Kytamer® PC (about 80% by weight active substance of chitosan pyrrolidone carboxylate (INCI name: Chitosan PCA), Amerchol) and Chitolam® NB/101.

The cosmetic agent may contain the at least one further firming fully synthetic polymer in a total amount of from about 0.01% by weight to about 20% by weight, more preferably from about 0.1% by weight to about 15% by weight, most preferably from about 0.5% by weight to about 10% by weight, each based on the total weight of the cosmetic agent.

The applicability of the cosmetic agent can be further increased by the use of small amounts of one or more polyhydric alcohols. Preferred cosmetic agents contain one or more C3 to C12 alkane-1,2-diols, more preferably C3 to C10 alkane-1,2-diols. The cosmetic agent particularly preferably contains propane-1,2-diol and caprylylglycol. These particularly preferred diols improve applicability, are quasi-nourishing and are suitable for forming a stable emulsion with the particularly preferred emulsifier. Based on the total weight of the cosmetic agent, in a preferred embodiment of the present disclosure, from about 0.01 to about 1.5% by weight, more preferably from about 0.02 to about 0.8% by weight of the alkane-1,2-diol is present in the cosmetic agent.

In a preferred embodiment of the present disclosure, the cosmetic agent contains at least one care oil and/or at least one care component, wherein vegetable care oil(s)/care component(s) are particularly preferred.

Oils are advantageous as care substances as they give the hair a silky shine and make the hair more resilient by being absorbed by the hair. Care oils, however, place higher demands on the product formulation, since these must be incorporated in a stable manner without showing disadvantageous settling or creaming with longer storage times of product containers.

According to a preferred embodiment of the present disclosure, the cosmetic agent comprises at least one care oil comprising a triglyceride of native origin. Particularly preferred are vegetable care oils from the group amaranth seed oil, argan oil, rice germ oil, baobab oil, Manetti oil, Marula seed oil, Yangu seed oil, rambutan oil, buckthorn oil, Monoi de Tahiti, tiger nut oil, Inca Inchi oil, avocado oil, cottonseed oil, cupuacu butter, cashew oil, thistle oil, peanut oil, jojoba oil, chamomile oil, coconut oil, pumpkin seed oil, linseed oil, macadamia oil, corn oil, almond oil, apricot kernel oil, poppy seed oil, evening primrose oil, olive oil, rapeseed oil, soybean oil, sunflower oil and wheat germ oil, in particular (−)-α-bisabolol, hydrogenated jojoba oil and/or coconut oil.

The agent may, for example, also contain at least one protein hydrolyzate and/or one of its derivatives as a care component. Protein hydrolyzates are product mixtures that are obtained by acid, alkaline or enzymatically catalyzed degradation of proteins. The term protein hydrolyzates is also understood to mean total hydrolyzates and individual amino acids and their derivatives and mixtures of different amino acids. The molecular weight of the protein hydrolyzates which can be used is between from about 75, the molecular weight for glycine, and about 200,000, preferably the molecular weight is from about 75 to about 50,000 and very particularly preferably from about 75 to about 20,000 daltons.

The agent may further contain at least one vitamin, a provitamin, a vitamin precursor and/or one of their derivatives as a care component. In this case, those preferred vitamins, provitamins and vitamin precursors are those which are usually assigned to the groups A, B, C, E, F and H. A particularly preferred care ingredient is D-panthenol.

The advantage of these care oils or care components is that they predominantly come from natural sources and thus represent energy- and resource-saving raw materials. According to the preferred embodiment, the cosmetic agent, based on the total weight, can contain from about 0.0005 to about 3% by weight, preferably from about 0.001 to about 2% by weight, and particularly preferably from about 0.05 to about 1% by weight of the sum of care oils and care component.

The cosmetic agent may further contain neutralizers or pH adjusters to adjust the pH. Examples of neutralizers used in cosmetic agents are NaOH or primary amino alcohols such as Aminomethyl Propanol (INCI), which is commercially available, for example, under the name AMP-ULTRA® PC, for example AMP-ULTRA® PC 2000.

In further preferred embodiments of the present disclosure, the cosmetic agent further contains preservatives, perfume and optionally further additives.

The cosmetic agent of the present disclosure can be formulated in the forms customary for the temporary shaping of hair, for example as a wax, paste, lotion, emulsion or clay. Preferably, the cosmetic agents are provided in cans or cups.

The cosmetic agents are preferably present as a foam. For this purpose, the cosmetic agents are packaged in a dispensing device, which is either a compressed gas container (“aerosol container”) additionally filled with a propellant or a non-aerosol container. The pressurized gas containers, with the aid of which a product is distributed by the internal gas pressure of the container via a valve, are defined as “aerosol containers”. In contrast to the aerosol definition, a container under normal pressure is defined as a “non-aerosol container”, with the aid of which a product is distributed by employing a mechanical action through a pumping or squeezing system.

Particularly preferably, the cosmetic agents are present as an aerosol foam in an aerosol container. The cosmetic agent therefore preferably additionally contains at least one propellant. In the embodiment as an aerosol foam, suitable propellants are, for example, selected from N₂O, dimethyl ether, CO₂, air, alkanes having from 3 to 5 carbon atoms, such as propane, n-butane, iso-butane, n-pentane and iso-pentane, and their mixtures. According to the embodiment of an aerosol foam, said alkanes, mixtures of said alkanes or mixtures of said alkanes with dimethyl ether are used as the sole propellant.

Preference is given to dimethyl ether, propane, n-butane, isobutane, pentane and mixtures thereof. The sizes of the foam bubbles and the respective size distribution can be set for a given spraying device via the quantity ratio of propellant to the other components of the preparations. When conventional aerosol containers are used, aerosol foam products preferably contain the propellant in amounts of from about 1 to about 35% by weight, based on the total product. Amounts of from about 2 to about 30% by weight, in particular from about 3 to about 15% by weight, are particularly preferred.

The present disclosure also relates to the cosmetic, non-therapeutic use of cosmetic agents as contemplated herein for the temporary shaping of keratinic fibers, in particular of human hair, and a method for the temporary deformation of keratinic fibers, in particular human hair, in which the cosmetic agent is applied on keratinic fibers.

### Embodiment 1

An aerosol mousse was prepared with the following recipe:

| | |
|---|---|
| Chitosan | 0.3% by weight |
| Lactic acid | 0.75% by weight |
| Gelatinized potato starch | 3.0% by weight |
| Sodium hydroxide | 0.25% by weight |
| Glycerol | 0.2% by weight |
| Cocoamidopropylbetaine | 0.3% by weight |
| PEG-40 Hydrogenated Castor Oil | 0.2% by weight |
| Sodium benzoate | 0.3% by weight |
| Water | 89.7% by weight |
| Propellant* | 5.0% by weight |

*Mixture of propane, n-butane, iso-butane and pentane

The mousse was filled in an aerosol container that meets the following technical parameters: Aluminum storage container with valve Product 522983 PV10697 from Pecision (Deutsche Prazisions-Ventil GmbH).

### Embodiment 2

To compare a variety of starches, the hair foam “Taft Volume Mousse” was set with different gelatinized starches before filling in an aerosol container:

| | | % by weight starch |
|---|---|---|
| Taft volume mousse | V1 | — |
| +Pea starch | V2 | 3.29 |
| +Wheat starch | V3 | 4.23 |
| +Corn starch | V4 | 3.29 |
| +Tapioca starch | V5 | 2.82 |
| +Potato starch | E1 | 1.88 |

### High Humidity Curl Retention Determination (HHCR)

Curl retention was measured at high humidity in hair strands that were treated with cosmetic agents V1 to V5 and E1.

For this purpose, standardized hair strands from Kerling (Item No. 827560) of the hair type “European Natural, Color 6/0” of a length (Lₘₐₓ) of 220 mm and a weight of 0.6 g were used. For preparation, the strands were washed with a 12.5% by weight sodium laureth sulfate solution. The strands of hair were dried overnight in a drying oven at 318 K.

0.18 g of the compositions were applied to one strand of hair and massaged in. The strand was then wound on a winder (Fripac-medis, Ø 7 mm, Type No. D-1203) and dried overnight at room temperature.

The winder was carefully removed and the strand hung. The lengths of the curls were each measured (L₀) and the strands placed in a climatic chamber. There they were stored at about 294 K and a relative humidity of about 85% over a period of about 24 h and then the lengths of the curls (Lₜ) measured again.

5 test strands per composition were treated and measured accordingly.

The high-humidity curl retention (HHCR) was calculated according to the following formula and the arithmetic mean for each composition was formed from the HHCR values of the 5 test strands:

$$H H C R = \frac{{L}_{max} - {L}_{t}}{{L}_{max} - {L}_{0}}$$

TABLE 1
Curl retention in high humidity (HHCR)

| Composition | HHCR |
|---|---|
| V1 | 46% |
| V2 | 59% |
| V3 | 62% |
| V4 | 69% |
| V5 | 76% |
| E1 | 74% |

### Stiffness

The stiffness of a polymer film formed on hair strands after the treatment with the cosmetic agents V1 to V5 and E1 was measured with the aid of the 3-point bending test.

In a dry strand of hair (Euro natural hair from Kerling, 826500 adhesive tress tight, glued on one side, total length 150 mm, free length 130 mm, width 20 mm, weight 1.8±0.2 g) 850 mg of a composition to be tested were massaged in with the fingers. The hair strand treated with the composition to be tested is in a straightened Teflon rack having a 20 mm diameter. The prepared strands were then dried and conditioned overnight at 21° C. and 50% relative humidity in the climatic room.

The conditioned strand was carefully removed from the Teflon rack. The resulting flat strand was placed on measuring blocks separated by 40 mm. The 3PB adapter of a universal testing device AMETEK LF Plus from AMETEK Precision Instruments Europe GmbH, product group Lloyd is mounted above this in the middle. The entire measurement was carried out in a climate chamber under constant climatic conditions at 21° C. and 50% relative humidity.

In order to achieve standardized starting conditions, the measurement started with the onset of a preload of 0.05 N. Then the strand was pressed by 15 mm at a speed of 500 mm min⁻¹, wherein the force required for this was measured. After this, the characteristic force K at the maximum deformation of 15 mm was recorded.

With this measurement method, the force Fmax can be used as a parameter to measure the stiffness of the polymer film and the degree of hold of the hairstyle hold generated by it.

10 strands were prepared and measured per test composition.

TABLE 2
Stiffness

| Composition | Fmax [N] |
|---|---|
| V1 | — |
| V2 | 3.27 |
| V3 | 2.80 |
| V4 | 3.73 |
| V5 | 3.78 |
| E1 | 4.30 |

The hair foam E1 as contemplated herein thus showed an exceptionally good combination of curl retention at high humidity and stiffness. It is particularly advantageous that this exceptional combination is achieved with a smaller amount of used, gelatinized starch.

While at least one exemplary embodiment has been presented in the foregoing detailed description, it should be appreciated that a vast number of variations exist. It should also be appreciated that the exemplary embodiment or exemplary embodiments are only examples, and are not intended to limit the scope, applicability, or configuration of the various embodiments in any way. Rather, the foregoing detailed description will provide those skilled in the art with a convenient road map for implementing an exemplary embodiment as contemplated herein. It being understood that various changes may be made in the function and arrangement of elements described in an exemplary embodiment without departing from the scope of the various embodiments as set forth in the appended claims.

1. A cosmetic agent for the temporary shaping of keratinic fibers, comprising:
from about 0.1 to about 50% by weight of potato starch, based on the total weight of the cosmetic agent.

2. The cosmetic agent according to claim 1, wherein the amount of potato starch is from about 1 to about 20% by weight, based on the total weight of the cosmetic agent.

3. The cosmetic agent according to claim 1, wherein the potato starch is modified and is selected from the group of gelatinized potato starch, alkaline modified potato starch and mixtures thereof.

4. The cosmetic agent according to claim 1, wherein the cosmetic agent further comprises natural starch of a species other than potato.

5. The cosmetic agent according to claim 4, wherein the amount of the natural starch of a species other than potato is from about 10% by weight to about 200% by weight of the amount of potato starch.

6. The cosmetic agent according to claim 1, wherein the cosmetic agent further comprises at least one care oil and/or at least one care component.

7. The cosmetic agent according to claim 1, wherein the cosmetic agent is present as a gel, wax, paste, lotion, emulsion or clay.

8. The cosmetic agent according to claim 1, wherein the cosmetic agent further comprises a propellant.

9. The cosmetic agent according to claim 1 further comprising a firming polymer.

10. A method for the temporary shaping of keratinic fibers comprising:
applying a cosmetic agent to the keratinic fibers, wherein the cosmetic agent comprises potato starch at from about 0.1 to about 50 weight percent, based on a total weight of the cosmetic agent.

11. The cosmetic agent according to claim 9 wherein the firming polymer is selected from polyvinylpyrrolidone, vinylpyrrolidone/vinyl acetate copolymer, vinylcaprolactam/vinylpyrrolidone (VP)/dimethylaminoethyl methacrylate copolymer (INCI), VP/dimethylaminopropylamine (DMAPA) Acrylate Copolymer (INCI) and mixtures thereof.

12. The cosmetic agent according to claim 9 wherein the firming polymer is an octylacrylamide/acrylates/butylaminoethyl methacrylate copolymer.

13. The cosmetic agent according to claim 9 wherein the firming polymer comprises chitosan and/or a chitosan derivative.

14. The method of claim 10 wherein applying the cosmetic agent comprises applying the cosmetic agent wherein the cosmetic agent comprises the potato starch at from about 1 to about 20 weight percent, based on the total weight of the cosmetic agent.

15. The method of claim 10 wherein applying the cosmetic agent comprises applying the cosmetic agent wherein the potato starch is selected from the group of gelatinized potato starch, alkaline modified potato starch and mixtures thereof.

16. The method of claim 10 wherein applying the cosmetic agent comprises applying the cosmetic agent wherein the cosmetic agent further comprises a natural starch from a species other than potato.

17. The method of claim 16 wherein applying the cosmetic agent comprises applying the cosmetic agent wherein the cosmetic agent comprises the natural starch from a species other than potato at from about 10% by weight to about 200% by weight, based on a total weight of the potato starch.

18. The method of claim 10 wherein applying the cosmetic agent comprises applying the cosmetic agent wherein the cosmetic agent further comprises a firming polymer.

19. The method of claim 18 wherein applying the cosmetic agent comprises applying the cosmetic agent wherein the firming polymer comprises chitosan and/or a chitosan derivative.

20. The method of claim 18 wherein applying the cosmetic agent comprises applying the cosmetic agent wherein the firming polymer is an octylacrylamide/acrylates/butylaminoethyl methacrylate copolymer.
